(19) United States
(12) Patent Application Publication
(10) Pub. No.: US 2025/0009551 A1
(43) Pub. Date: Jan. 9, 2025

(54) URINE COLLECTING DEVICE

(71) Applicant: Chen Chu HSIEH, Miaoli County (TW)

(72) Inventor: Chen Chu HSIEH, Miaoli County (TW)

(21) Appl. No.: 18/378,745

(22) Filed: Oct. 11, 2023

(30) Foreign Application Priority Data
Jul. 3, 2023 (TW) 112206848

(51) Int. Cl. A61F 5/44; A61F 13/47

(52) U.S. Cl. CPC A61F 5/4408; A61F 5/4401; A61F 13/47

(57) ABSTRACT

A urine collecting device comprises: a urine bag including an opening; a side surface having a notch, wherein the notch extends from the opening; and a bottom edge opposite to the opening, a girdle with a hook and loop fastener: a strong clamp; and a sanitary pad; wherein the urine bag is worn on a body of a user in a manner of aligning a bottom end of the notch with a lower edge of a scrotum of the user and the opening passing through the girdle with the hook and loop fastener from an inside formed by the girdle with the hook and loop fastener surrounding a waist of the user so that the urine bag is fixed on the waist of the user by the girdle with the hook and loop fastener, wherein the urine bag is fixed on the girdle with the hook and loop fastener by the strong clamp, and wherein the sanitary pad is provided at the bottom end, and the bottom end is an end of the notch away from the opening.

### FIELD OF THE INVENTION

The present invention relates to a urine collecting device.

### BACKGROUND OF THE INVENTION

For those with uriniparous inconvenience or uriniparous problems of bedfast patients, how to deal with multiple urinations during the day and night becomes a troublesome problem. The existing process usually adopts two approach such as wearing a diaper or wearing a uriniparous tool.

However, when wearing a conventional uriniparous tool, a uriniparous tool that loosely fits a uriniparous engaging structure encased on a penis or a radial gap between the uriniparous engaging structure and the urinating connecting structure becoming larger due to a physiological shrinkage of the penis and a urethral orifice axially closer to the outer edge of the interface of the uriniparous engaging structure eventually causes urine leakage. In comparison, it feels very uncomfortable due to the close contact with the penis when a uriniparous tool that tightly fits a uriniparous engaging structure on the penis is worn. It is difficult for a user to wear it for a long time.

Wearing a diaper will bring a sense of moisture to the skin. The user also feels uncomfortable when it is worn for a long time. Such discomfort makes it impossible to live a normal daily life during the day, and the quality of sleeping at night is also affected so that it is difficult to fall asleep. Moreover, a diaper has a necessity that must be changed frequently. In addition to assisting in changing it during the day, the caregiver must also adapt to change it at night so that neither the user nor the caregiver can sleep continuously. Therefore, high changing frequency not only increases material cost, but also increases personnel cost.

Therefore, how to solve the problem of uriniparous inconvenience or uriniparous problems of bedfast patients, improve the comfort of urination, and reduce costs are problems that practitioners in the related fields must further solve.

### SUMMARY OF THE INVENTION

A first aspect of the present invention provides a urine collecting device, comprising: a urine bag including an opening; a side surface having a notch, wherein the notch extends from the opening; and a bottom edge opposite to the opening, a girdle with a hook and loop fastener: a strong clamp; and a sanitary pad; wherein the urine bag is worn on a body of a user in a manner of aligning a bottom end of the notch with a lower edge of a scrotum of the user and the opening passing through the girdle with the hook and loop fastener from an inside formed by the girdle with the hook and loop fastener surrounding a waist of the user so that the urine bag is fixed on the waist of the user by the girdle with the hook and loop fastener, wherein the urine bag is fixed on the girdle with the hook and loop fastener by the strong clamp, and wherein the sanitary pad is provided at the bottom end, and the bottom end is an end of the notch away from the opening.

A second aspect of the present invention further comprises a mesh elastic bandage for fixing a portion of the urine bag adjacent to the bottom edge to a calf of the user to solve the problems mentioned in the above prior art.

A third aspect of the present invention further comprises a contracted rope for tightening a portion of the urine bag adjacent to the bottom edge.

A fourth aspect of the present invention further comprises another strong clamp for fixing the tightened bottom edge on the girdle with the hook and loop fastener or on the body of the user.

A fifth aspect of the present invention is that wherein the urine bag is a high-density polyethylene (HDPE) plastic bag.

The above-mentioned aspect of the present invention solves the uriniparous inconvenience or uriniparous problems of bedfast patients, improves the comfort of urination, and reduces the material cost and the personnel cost.

### BRIEF DESCRIPTION OF THE DRAWINGS

FIG. 1 illustrates a three dimensional view of a urine bag according to an embodiment of the present invention.

FIG. 2 illustrates a front perspective view of a urine collecting device used on a body of a user according to an embodiment of the present invention.

FIG. 3 illustrates a partial cross-sectional view of the urine collecting device according to an embodiment of the present invention along a line segment A-A of FIG. 2.

FIG. 4 illustrates a side perspective view of a urine collecting device used on a body of a user wearing a pant according to an embodiment of the present invention.

FIG. 5 illustrates a side perspective view of a urine collecting device used on a body of a user according to an embodiment of the present invention.

FIG. 6 illustrates a side view of a urine collecting device used on a body of a user according to an embodiment of the present invention.

### DETAILED DESCRIPTION OF THE INVENTION

Hereafter, a plurality of embodiments of the present invention will be disclosed with drawings. For the purposes of a clear description, many practical details will together be described below. However, it should be understood that these practical details are not for restricting the present invention. That is to say, in parts of the embodiments of the present invention, these practical details are not necessary. In addition, for the sake of simplifying figures, some conventional structures and elements in figures will be shown in a simple 10 schematic manner.

The embodiment will be described in detail with reference to the drawings. Note that multiple embodiments of the present invention will be disclosed in the following drawings. For the sake of clarity, many practical details will be explained in the following description. However, it should be understood that these practical details should not be used to limit the present invention. That is to say, in some embodiments of the present invention, these practical details are not necessary. In addition, for the purpose of simplifying the drawings, some commonly used structures and components are illustrated in a simple schematic manner in the drawings.

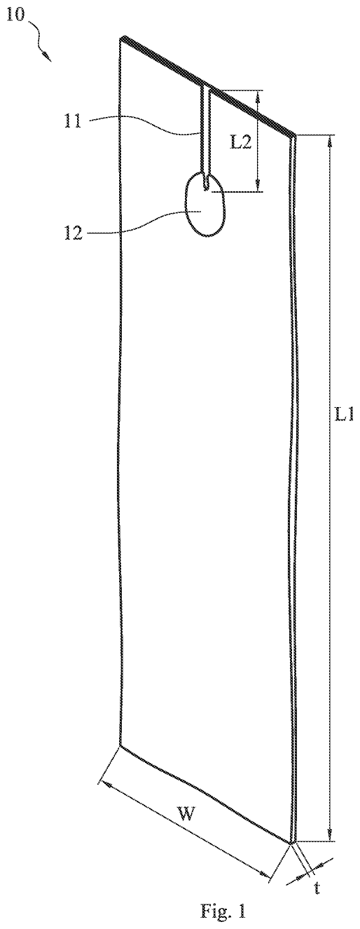

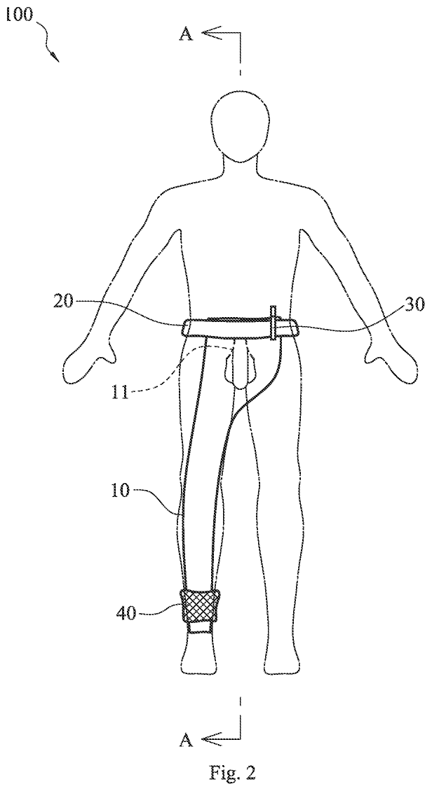

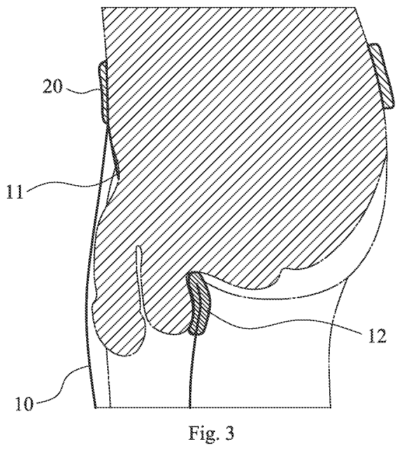

FIG. 1 illustrates a three dimensional view of a urine bag according to an embodiment of the present invention. FIG. 2 illustrates a front perspective view of a urine collecting device used on a body of a user according to an embodiment of the present invention. FIG. 3 illustrates a partial cross-sectional view of the urine collecting device according to an embodiment of the present invention along a line segment A-A of FIG. 2. As shown in FIGS. 1, 2 and 3, in an embodiment of the present invention, a urine collecting device 100 is provided, and comprises a urine bag 10; a girdle with a hook and loop fastener 20; a strong clamp 30; and a sanitary pad 12. The urine bag 10 includes an opening; side surfaces; and a bottom edge. The side surface has a notch 11. The notch 11 extends from the opening. The bottom edge and the opening are opposite to each other. The urine bag 10 has a length L1, a width W, and a thickness t. The notch 11 of the urine bag 10 has a length L2.

In an embodiment of the present invention, the urine bag 10 is worn by aligning a bottom end of the notch 11 with a lower edge of a scrotum of a user so that a penis and the scrotum of the user are collected into the urine bag 10 together. The advantage of this embodiment is that the notch 11 of the urine bag 10 is contacted with the scrotum, rather than the penis, of the user in a loose and untightened manner, which will not cause physical discomfort to the user. In addition to the loose and untightened manner of contact, the urine bag 10 has the very thin thickness (t) and there is a considerable flexibility for the plastic material thereof. Therefore, it is advantageous to wear the urine collecting device 100 of the present invention for a long time.

Moreover, in this embodiment, since the bottom end of the notch 11 of the urine bag 10 is aligned with the lower edge of the scrotum of the user, the notch 11 is further away from a position of a urethral orifice of the penis. Therefore, there is no urine leakage problem of a loose-fitting uriniparous tool or due to physiological contraction of the penis.

Following the technical content of the above embodiment, before the tightening action of the girdle with the hook and loop fastener 20 surrounding a waist of the user, the opening passes through the girdle with the hook and loop fastener 20 from an inside formed a circle surrounded by the girdle with the hook and loop fastener 20 in a direction from lower limbs to upper limbs of the user. Then, the tightening action of the girdle with the hook and loop fastener 20 mentioned above is completed. The urine bag 10 is worn on the body of the user in the above manner, and is fixed on a waist of the user by the girdle with the hook and loop fastener 20.

In an embodiment of the present invention, considering that the urine bag 10 needs to carry a weight of urine for several kilograms, the urine bag 10 is also fixed on the girdle with the hook and loop fastener 20 by a strong clamp 30.

In an embodiment of the present invention, considering a more advantageous manner to wearing the urine collecting device 100 of the present invention for a long time, the sanitary pad 12 is provided at a position of a bottom end of the notch 11, that is, an end of the notch 11 away from the opening. That is to say, the sanitary pad 12 is folded in half and adhered along and on an end in the notch 11 formed by converging in a shape similar to “V”. In this way, in addition to the side edges of the notch 11 contacting the scrotum of the user in a loose and untightened manner, the lower edge of the notch 11 further enhances the comfort of the user to wear the urine collecting device 100 of the present invention for a long time due to the provision of the sanitary pad 12.

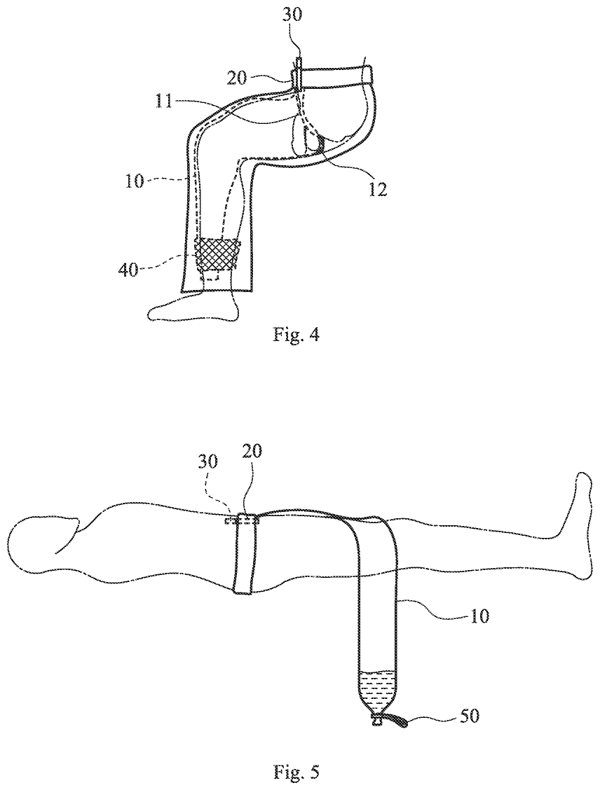

FIG. 4 illustrates a side perspective view of a urine collecting device used on a body of a user wearing a pant according to an embodiment of the present invention. In an embodiment of the present invention, the use of the present invention does not affect an appearance of the clothing of the user. For example, regarding daily clothes worn by the user, outdoor activities, working, and the like, in order to prevent daily activities and working of the user from being affected by the behavior of urination, as shown in FIGS. 2 and 4, in an embodiment of the present invention, it further comprises a mesh elastic bandage 40 for fixing a portion of the urine bag 10 adjacent to the bottom edge to a calf of the user. Since both ends (the opening and the portion adjacent to the bottom edge) of the urine bag 10 are fixed, in a condition that the user wears the daily clothes, he can naturally accommodate the urine collecting device 100 within the daily clothes. In this way, the daily activities and working of the user will not be affected by the behavior of urination. In particular, as shown in FIG. 4, when the user is in a condition of a sitting posture, such as working, eating, talking, and the like, the urine collected in the urine bag 10 at the height of the calf of the user will not flow back to cause a leakage problem due to the sitting posture.

FIG. 5 illustrates a side perspective view of a urine collecting device used on a body of a user according to an embodiment of the present invention. In an embodiment of the present invention, it further comprises a contracted rope 50 for tightening a portion of the urine bag 10 adjacent to the bottom edge. In a condition that the user is in a lying posture, for example, bedfast need due to physical discomfort, resting during the day, sleeping at night, and the like, in order to improve the comfort of the lying posture and the quality of resting and sleeping, there is no need to fix a portion of the urine bag 10 adjacent to the bottom edge on the body of the user. Therefore, it is only necessary to naturally hang the portion of the urine bag 10 adjacent to the bottom edge tightened by the contracted rope 50 down from the side of the bed until it touches the ground to facilitate the collection of urine. In this way, the weight of the urine bag 10 is counterbalanced by a reaction force from the ground, thereby allowing the user to pay his attention to resting and sleeping without bearing the weight of the urine bag 10.

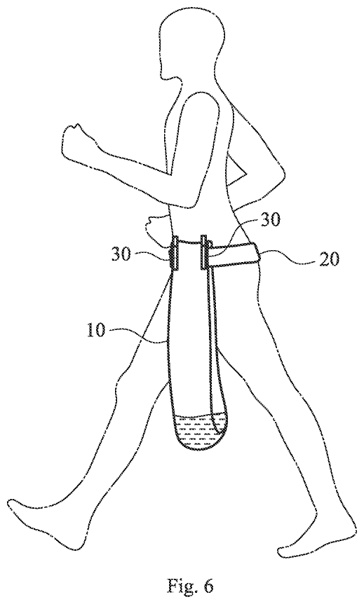

FIG. 6 illustrates a side view of a urine collecting device used on a body of a user according to an embodiment of the present invention. In an embodiment of the present invention, it further comprises another strong clamp 30 for fixing the tightened bottom edge on the girdle with the hook and loop fastener 20 or on the body of the user. In a condition of a duration of a transition for the user from daily activities and working to a lying posture or other than daily activities, working, and a lying posture, for example, after a completion of taking a bath, before going to bed, indoor activities at night, and the like, in order to avoid the portion of the urine bag 10 adjacent to the bottom edge from dragging on the ground so as to cause the urine bag 10 to be broken with a leakage problem, fixing the tightened bottom edge on the girdle with the hook and loop fastener 20 or on the body of the user allows the user to perform indoor activities before going to bed and at night. In this condition, there is no need to hold the urine bag 10 by hand and the urine bag 10 will not be dragged on the ground. In this embodiment, the user can use a bath towel (for example, the length and width are 1700 mm and 700 m respectively) to wrap around the lower body to cooperate with the implementation of this embodiment. In this embodiment, one of the example of being on the body of the user can be on a collar of the user.

In an embodiment of the present invention, the urine bag may be a high-density polyethylene (HDPE) plastic bag. However, this material is only an example and is not intended to limit the present invention. For example, it can also be other high-density plastic bags. The material of the urine bag can be flexibly selected depending on the spatial size and the actual needs for a person of ordinary skill in the art of the present invention.

In an embodiment of the present invention, the specification of the urine bag 10 can be divided into two types such as daytime and nighttime. During daytime, for example, the length (L1), width (W), and thickness (t) respectively being 1600 mm, 300 mm, and 0.02 mm can be used. During nighttime, for example: the length (L1), width (W) and thickness (t) respectively being 1300 mm, 300 mm, and 0.02 mm can be used. The length (L2) of the notch 11 of the urine bag 10 may be 300 mm to 500 mm. However, this specification is only an example and is not intended to limit the present invention. For example, the bodies of European, American, and Asian users are obviously different. Therefore, the width (W) can also be in the range of 150 mm to 300 mm or other ranges, and the length (L1) can also be in the range of 1300 mm to 1600 mm or other ranges. The specification of the urine bag 10 and the notch 11 can be flexibly selected depending on the spatial size and the actual needs for a person of ordinary skill in the art of the present invention.

In an embodiment of the present invention, the sanitary pad 12 can be, for example, breathable and ultra-thin sanitary pad of Carnation. The specification of the girdle with the hook and loop fastener 20 can be used, for example: the length, width and thickness respectively being 1350 mm, 50 mm, and 2 mm. The specification of the strong clamp 30 can be, for example, a clamp sufficient to fix the urine bag 10 carrying several kilograms of urine. The specification of the mesh elastic bandage 40 can be used, for example: Rifeng mesh elastic bandage No. 5. The contracted rope 50 can be made of, for example, polypropylene (PP). However, the above specifications are only examples and are not intended to limit the present invention. As mentioned above, the body shapes of European, American, and Asian users are obviously different. Therefore, the specification of the sanitary pad 12, the girdle with the hook and loop fastener 20, the strong clamp 30, the mesh elastic bandage 40 and the contracted rope 50 can be flexibly selected depending on the spatial size and the actual needs for a person of ordinary skill in the art of the present invention.

In an embodiment of the present invention, the following steps may be included:
1. Properly fastening the girdle with the hook and loop fastener 20 to the user's waist;
2. Cutting out a notch 11 of 400 mm to 500 mm from the urine bag 10;
3. Bonding the sanitary pad 12 to the notch 11;
4. Smearing some oil-soluble vaseline inside and outside the pad 12 to increase lubricity;
5. Aligning the notch 11 with the male user's scrotum and encasing the scrotum;
6. Passing the opening of the urine bag 10 through the girdle with the hook and loop fastener 20 between the user's waist and the girdle with the hook and loop fastener 20;
7. After folding the opening of the urine bag 10 that has passed through the girdle with the hook and loop fastener 20 from top to bottom at the periphery of the girdle with the hook and loop fastener 20, clamping the folded part with the girdle with the hook and loop fastener 20 to the user's waist by using the strong clamp 30.

In an embodiment of the present invention, after the urine bag 10 is filled with urine, it can be divided into one-time-use mode and multiple-time-use mode. The multiple-time-use mode is to pour the collected urine from the opening of the urine bag 10 into the toilet so as to repeatedly use the urine bag 10. The one-time use mode is to cut a small kerf on the urine bag 10 above the toilet so that the filled urine can flow out from the kerf on the urine bag 10 into the toilet.

Compared with diapers, the embodiments of the present invention are convenient and simple to be used, and does not require frequent replacement. The user's suffering from being soaked in urine in diapers is mitigated. Sleeping at night can last until dawn.

From the perspective of economic cost, disposable diapers are consumables and expensive, and they impose economic burden the on users. If they are replaced with the present invention, the economic burden can be mitigated by the present invention.

Since the present invention can be repeatedly used after disinfection, the usage of the present invention comprises but is not limited to patients who are bedfast or have uriniparous inconvenience. For users who are inconvenient to go to the toilet due to long working hours, such as those who are drivers of transportation or workers in clean rooms, using the present invention does not require holding their urine and allows them to work comfortably all day long.

The structure of the embodiments of the present invention is simple. Compared with the conventional uriniparous device, the present invention does not require a urethral catheter, additional structures specifically used to engage uriniparous organs of a human body, structures for connections between numerous structures and for leak-proof. Compared with the conventional diapers, the present invention does not cause discomfort after urination, and does not require structures such as absorbent materials and sensors for sensing humidity.

The above-mentioned embodiments of the present invention solve the problem of uriniparous inconvenience or uriniparous problems of bedfast patients, improves the comfort of urination, and reduces the material cost and the personnel cost. Moreover, the structure of the conventional uriniparous tool is simplified so that the user does not leak urine during day and night activities, work, resting, and sleeping, and there is no necessity for a caregiver to change it frequently. Therefore, the quality of resting, sleeping and life of users and caregivers is also improved.

Although the present invention has been disclosed above with embodiments, these embodiments do not limit the present invention. People skilled in this art can make various modifications and retouches without departing from the spirit and scope of the invention. Accordingly, the scope protected by the present invention shall be based on the content defined by the appended claims.

What is claimed is:

1. A urine collecting device, comprising:
a urine bag, including:
an opening;
a side surface having a notch, wherein the notch extends from the opening; and
a bottom edge opposite to the opening,
a girdle with a hook and loop fastener:
a strong clamp; and
a sanitary pad;
wherein the urine bag is worn on a body of a user in a manner of aligning a bottom end of the notch with a lower edge of a scrotum of the user and the opening passing through the girdle with the book and loop fastener from an inside formed by the girdle with the hook and loop fastener surrounding a waist of the user so that the urine bag is fixed on the waist of the user by the girdle with the hook and loop fastener,
wherein the urine bag is fixed on the girdle with the hook and loop fastener by the strong clamp, and
wherein the sanitary pad is provided at the bottom end, and the bottom end is an end of the notch away from the opening.

2. The urine collecting device of claim 1, further comprising a mesh elastic bandage for fixing a portion of the urine bag adjacent to the bottom edge to a calf of the user.

3. The urine collecting device of claim 1, further comprising a contracted rope for tightening a portion of the urine bag adjacent to the bottom edge.

4. The urine collecting device of claim 3, further comprising another strong clamp for fixing the tightened bottom edge on the girdle with the hook and loop fastener or on the body of the user.

5. The urine collecting device of claim 1, wherein the urine bag is a high-density polyethylene (HDPE) plastic bag.
